(19) United States
(12) Patent Application Publication
(10) Pub. No.: US 2021/0293700 A1
(43) Pub. Date: Sep. 23, 2021

(54) DETECTING COMPOSITION OF NON-HOMOGENIZED FLUIDS

(71) Applicant: PerkinElmer Health Sciences B.V., Drachten (NL)

(72) Inventors: Patrick Cree, Drachten (NL); Martin Land, Drachten (NL); Anne Gerben Terpstra, Drachten (NL); Hendrick De Vries, Drachten (NL)

(21) Appl. No.: 17/208,410

(22) Filed: Mar. 22, 2021

Related U.S. Application Data
(60) Provisional application No. 62/992,736, filed on Mar. 20, 2020.

(51) Int. Cl. G01N 21/3577; G01N 33/06; G01N 15/02

(52) U.S. Cl. CPC G01N 21/3577; G01N 2021/3595; G01N 15/0205; G01N 33/06

(57) ABSTRACT

Provided herein are methods, systems, and apparatus related to sensing and measuring various components in non-homogenized solutions. Also provided herein are systems for sensing a property of a solution containing one or more components. Also provided herein are methods for determining fat content in a non-homogenized solution.

### CROSS-REFERENCE TO RELATED APPLICATIONS

This application claims priority to U.S. Provisional Patent Application No. 62/992,736, filed Mar. 20, 2020, which is incorporated herein by reference in its entirety.

### TECHNICAL FIELD

This document describes technology for sensing the composition of non-homogenized fluids, including by the use of infrared sensors.

### BACKGROUND

Fluids can be made up of multiple components. A homogeneous mixture is a solid, liquid, or gaseous mixture that has the same proportions of its components throughout any given sample. Conversely, a heterogeneous mixture has components whose proportions vary throughout the sample.

Milk is a nutrient-rich liquid food product produced by mammals, and is often the primary source of nutrition for infant animals before they are old enough to digest other types of food. Milk is an example of a fluid that is naturally heterogeneous, but can be subjected to a homogenizing process to homogenize the milk into a homogeneous state.

### SUMMARY

The methods, systems and apparatuses described herein are based, in part, on the discovery that fluid sensing can be performed on non-homogenized solutions using Fourier-transform infrared spectroscopy and infrared sensors to sense energy in the mid-infrared range (MIR,). For example, by utilizing Fourier-transform infrared spectroscopy using infrared sensors able to sense energy in the mid-infrared range (MIR,) non-homogenized milk can be sensed. This sensing can then produce useful measures such as measures of fat within non-homogenized milk. By being able to sense using milk that have not had to undergo homogenization, this technology advantageously can operate without the cost, space requirements, and complexity of a homogenizer. For example, a milk-analyzing device using this technology can be smaller, less expensive, easier to use, less prone to failure, and more portable than a milk analyzer that includes a homogenizer. The methods, systems, and apparatuses described herein provide several advantages over the prior art. First, spectral analyzers without a homogenizer, whether of diagnostic or assistive purposes, can decrease the time and costs of the methods and systems described herein as compared to spectral analyzers with a homogenizer. Solution/spectral analyzers, which include a homogenizer, can be prohibitively expensive, especially for low-gross income companies. This is due to the fact that homogenizers tend to be fragile, break easily, wear out quickly, and need to be replaced often. In addition, each time a homogenizer is repaired or replaced, on a solution analyzer, the analyzer needs to be recalibrated using reference solutions. This recalibration takes time to run all of the reference samples and is very costly due to the purchase of the reference samples. In some implementations, the methods, systems, and other techniques described herein can provide professionals with a solution-analyzing device that is smaller, less expensive, easier to use, less prone to failure and more portable than a solution analyzer with a homogenizer. Spectral/solution analyzers with a homogenizer also require a higher volume of solution in order to determine the amount of components within the solution it is analyzing, due to loss of sample solution during the homogenization step. Homogenizers can also influence calibration in a negative manner by introducing additional scatter.

In one aspect, this disclosure features methods for measuring a component of a non-homogenized solution, the method including: receiving, at a computer system, a datastream of sensed infrared energy generated by irradiating a sample of the non-homogenized solution with infrared energy, and sensing the infrared energy emitted from the irradiated sample; determining, by the computer system, at least one of an infrared absorption spectrum and an infrared emission spectrum based on the datastream; determining, by the system, a measured value of the component in the sample based on the one or more determined spectrum; generating, by the computer system, an adjustment factor based on the one or more determined spectrum; adjusting the measured value based on the adjustment factor to generate an adjusted value; determining, by the system, a correction factor based on a selected particle size or particle scatter associated with the component in the sample; modifying, by the computer system, the adjusted value using the correction factor, to generate a corrected value of the component; and, outputting, by the computer system, information identifying the corrected value of the component, wherein the corrected value indicates a measurement of the component in the non-homogenized solution.

In some embodiments, the selected particle size is based on a mean particle size of a predetermined proportion of particles of the component in at least a portion of the sample.

In some embodiments, the mean particle size or particle scatter is determined based on at least one of the infrared absorption spectrum and the infrared emission spectrum.

In some embodiments, the selected particle size is determined based on a predetermined proportion of particles of the component in at least a portion of the sample having a mean diameter less than the selected particle size. In some embodiments, the predetermined proportion is between twenty percent and one hundred percent. In some embodiments, the predetermined proportion is ninety percent.

In some embodiments, the correction factor is based on a relationship between an error measurement model and the selected particle size or the particle scatter, where such relationship corresponds to a reference model associated with the component. In some embodiments, the relationship is a linear relationship.

In some embodiments, generating an adjustment factor based on the one or more determined spectrum further includes obtaining a reference spectrum associated with the component and comparing the one or more determined spectrum to the reference spectrum to generate the adjustment factor. In some embodiments, comparing the one or more determined spectrum to the reference spectrum includes performing a linear least squares regression. In some embodiments, the reference spectrum is associated with measurements of the component in a homogenized solution.

In some embodiments, the non-homogenized solution is non-homogenized milk and the component is fat.

In some embodiments, generating an adjustment factor based on the one or more determined spectrum further includes obtaining a reference spectrum associated with the component and comparing the determined spectrum to the reference spectrum to generate the adjustment factor wherein the reference spectrum is based on one or more mathematical models including Fat A model, Fat B model, Fat C model, Fat D model, or Fat PLS model. In some embodiments, the correction factor is based on a relationship between error measurement and particle size, wherein the relationship is associated with a fat model spectrum comprising at least one of a Fat A model, a Fat B model, a Fat C model, a Fat D model, or a Fat PLS model. In some embodiments, particle size is based on a mean diameter of at least some of the particles in the solution. In some embodiments, the particle size is D90.

In some embodiments, the sample is a solution of non-homogenized milk, and the component includes one or more of fat, protein, total protein, true protein, lactose, non-protein nitrogen, solids, or non-fat solids, or any combinations thereof.

In another aspect, this disclosure features methods for determining fat content of a non-homogenized solution, the method implemented by a system including at least one computer, the method including: receiving a datastream of sensed infrared energy generated by irradiating a sample of the non-homogenized solution and sensing the infrared energy from the irradiated sample; determining, at least one of an infrared absorption spectrum and an infrared emission spectrum based on the received datastream of sensed infrared energy; selecting a reference spectrum for fat content based on the non-homogenized solution; comparing at least one of the infrared absorption spectrum and the infrared emission spectrum to the reference spectrum to determine an adjusted value of an amount of fat in the non-homogenized solution; determining a particle size of the fat content based on at least one of the infrared absorption spectrum and the infrared emission spectrum; based on the determined particle size, computing a correction factor; and determining the fat content in the sample by applying the correction factor to the adjusted value.

In some embodiments, determining a particle size includes determining a fat particle size within a predetermined proportion of fat particles in at least a portion of the sample. In some embodiments, the predetermined proportion is ninety percent.

In some embodiments, selecting a reference spectrum includes selecting at least one fat model spectrum associated with a Fat A model, a Fat B model, a Fat C model, a Fat D model, and a Fat PLS model.

In some embodiments, computing a correction factor includes selecting an error measurement model associated with the at least one selected fat model spectrum.

In some embodiments, the non-homogenized solution is one of an animal dairy product or a non-animal milk product. In some embodiments, the animal dairy product includes at least one of raw milk, milk, cream, ice cream, yogurt, cheese, or any combinations thereof. In some embodiments, the animal dairy product includes milk from at least one of a cow, a sheep, a camel, a buffalo, a goat, and a human.

In another aspect, this disclosure features systems for sensing a property of a non-homogenized solution containing one or more components, the system including: a sample chamber configured to receive a sample of the non-homogenized solution; an infrared energy source configured to, when energized, irradiate the sample with infrared energy; an infrared sensor including: a sensing element positioned to receive infrared energy emitted from the irradiated sample and configured to generate a datastream based on the received infrared energy; and a controller including a processor and a memory, the controller being in data communication with the infrared sensor, the controller configured to: determine at least one of an infrared absorption spectrum and an infrared emission spectrum from the datastream; process the one or more measured spectrum to compute a measured value of an amount of a component of the sample; process the one or more determined spectrum to generate an adjustment factor; adjust the measured value based on the adjustment factor to generate an adjusted value; determine a correction factor based on a selected particle size associated with the component in the sample; and, modify the adjusted value using the correction factor, to generate a corrected value of the component.

In some embodiments, the controller is further configured to: compare the corrected value of the component to a ruleset to identify an operation defined by the ruleset; and responsive to the identification of an operation, issue a command to cause the operation to occur. In some embodiments, the operation includes at least one of: initiating operation of a device that manufactures a product using the non-homogenized solution; actuating a transfer device that transfers the non-homogenized solution from an initial location to a destination location; transmitting a first data record over a data network, the data record created based on the corrected value; and causing storing of a second data record to a computer-readable destination.

Unless otherwise defined, all technical and scientific terms used herein have the same meaning as commonly understood by one of ordinary skill in the art to which this invention belongs. Methods and materials are described herein for use in the present invention; other suitable methods and materials known I the art can also be used. The materials, methods, and examples are illustrative only and not intended to be limiting. All publications, patent applications, patents, and other references mentioned herein are incorporated by reference in their entirety. In case of conflict, the present specification, including definitions, will control.

It will be further understood that the terms “includes,” “comprises,” “including” and/or “comprising,” when used in this specification, specify the presence of stated features, integers, operations, elements, and/or components, but do not preclude the presence or addition of one or more other features, integers, operations, elements, components, and/or groups thereof. The recitations of numerical ranges by endpoints include all numbers subsumed within that range (e.g., 1 to 5 includes, 1, 1.5, 2, 2.75, 3, 3.8, 4, 5, etc.).

For the terms “for example” and “such as,” and grammatical equivalences thereof, the phrase “and without limitation” is understood to follow unless explicitly stated otherwise. As used herein, the term “about” is meant to account for variations due to experimental error. As used herein, the singular forms “a,” “an,” and “the” are used interchangeably and include plural referents unless the context clearly dictates otherwise.

As used herein, the term “non-homogenized” includes “unhomogenized” and “not homogenized” as well as other equivalent expressions. Homogenization, in general, shall be understood to be a process to reduce a substance to substantially uniform size particles and to evenly distribute such particles. Those of ordinary skill understand that there are many ways to perform homogenization, and more well-known process include the use of, e.g., mechanical, acoustical, optical, and/or ultrasonic devices. As an example, when the sample solution is milk, homogenization is often understood to be a process that mixes and disperses milkfat, and this process is often performed using a high-pressure procedure to break the milkfat into smaller particles. Accordingly, as used herein, non-homogenized shall be understood to apply to, and/or be characteristic of, a substance that has not been subject to homogenization or a homogenization process.

As used herein, “substantially uniform size particles” in relation to a solution, shall mean a solution or sample in which the different constituents or components of the solution/sample may not be visibly discerned with the naked eye.

Also herein, the recitations of numerical ranges by endpoints include all numbers subsumed within that range (e.g., 1 to 5 includes, 1, 1.5, 2, 2.75, 3, 3.8, 4, 5, etc.).

The details of one or more embodiments of the present disclosure are set forth in the accompanying drawings and the description below. Other features, objects, and advantages of the disclosure will be apparent from the description and drawings, and from the claims.

### DESCRIPTION OF DRAWINGS

FIG. 1 is a conceptual diagram of an example system that can be used for determining a measure of a component of a fluid sample, recording and processing spectral data representative of milk components, and using the data to generate a value of individual milk components.

FIG. 2 is a flowchart of an example process used to perform operations on a fluid.

FIG. 3 is a flowchart of an example process for determining readings.

FIG. 4 is a flowchart of an example process for calibration.

FIG. 5 is a schematic diagram that shows an example of a computing system.

Like reference symbols in the various drawings indicate like elements.

### DETAILED DESCRIPTION

Measurement of certain components or aspects of non-homogenized samples, mixtures or solutions can be difficult to accurately measure. For example, measurements of fat content, lactose, and urea in non-homogenized milk are considered unreliable, particularly when compared to the accuracy of such measurements when performed on the sample, mixture or solution after the costly and disruptive homogenization process. The disclosed methods, systems, and apparatus thus seek to increase the accuracy of measuring a component of a non-homogenized sample through the computation and use of a correction factor that may be applied to measurements of the non-homogenized sample. The correction factor may be derived from and/or based on one or more representative models related to the measured component.

Accordingly, disclosed herein are computer-implemented methods, systems, and apparatus related to sensing and measuring various components in non-homogenized solutions or samples. The methods can be performed by systems comprising one or more computers or microprocessor-driven devices (e.g., tablet, phone, laptop, desktop, etc.) in one or more locations. In some aspects, the systems have one or more microprocessors (“processors”) and one or more computer-readable media encoded with instructions that, when executed by the one or more processors, cause the processors to perform the disclosed methods. Some aspects include just the computer-readable media encoded with instructions that cause performance of the method when executed.

The technology of fluid sensing is varied and sophisticated, and includes, for example, utilizing Fourier-transform infrared spectroscopy and infrared sensors to sense energy in a given frequency/wavenumber range, e.g., the mid-infrared range (“MIR”), to analyze or sense fluid components. These technologies are typically incorporated into a spectral analyzer. In the present disclosure, the example non-homogenized sample, mixture, or solution that is non-homogenized milk, however the present disclosure is not so limited to milk and can be applied to other non-homogenized solutions. In the example of non-homogenized milk, sensing by various technologies can produce useful measures of the sample components, such as measures of fat within the non-homogenized milk. By measuring non-homogenized milk that has not had to undergo homogenization, this technology of the present disclosure can advantageously operate without the cost, space requirements, and complexity of a homogenizer. For example, a milk-analyzing device using the techniques of the present disclosure can be smaller, less expensive, easier to use, less prone to failure, and more portable than a milk analyzer that includes a homogenizer.

According the present disclosure, one or more components of a non-homogenized fluid solution are sensed. For example, a sample of non-homogenized milk can be received and subjected to infrared energy, which is then sensed by corresponding sensors. From this sensing/sensor measurements, a measure of one or more components of the fluid is calculated. In the non-limiting example of non-homogenized milk, this technology can be used to determine, for example, the percentage of fat within the sample of non-homogenized milk, although an absolute value of the amount of fat may also be determined in some embodiments. As such, the non-homogenized milk may be advantageously sensed without the use of a homogenizer or homogenization process, thereby avoiding the use of or need for a homogenizing device.

In some embodiments, the disclosure herein provides processes for improving the accuracy of a representation of an amount of one or more components present in a sample of a non-homogenized solution, such as, for example, milk. Typically, when a component, such as fat content, is measured in milk, the measurement is made on homogenized milk. That measurement may be compared to an appropriate milk fat model and corrected using, for example, a regression model. In some instances, the correction is a linear correction.

Those of ordinary skill will understand that there are many different milk fat models that may be derived using and/or based on different known or “reference” samples of a substance. For example, different fat models, some of which may be mathematically derived, may take into account different types or properties of milk samples (e.g., dairy milk, heavy cream, milk storage characteristics, and/or season/time of year of milk extraction). Milk is made up of multiple components, including, but not limited to: fat, protein, total protein, true protein, lactose, non-protein nitrogen (typically in the form of urea), solids, or non-fat solids. Based on these measurements of milk contents, e.g., fat content, stored milk associated with a given sample analyzed pursuant to the disclosed methods, systems, and apparatus, may be designated and/or routed for a particular purpose (e.g., cheese, livestock, yogurt, etc.). In some embodiments, levels of these components are used to determine various factors related to dairy farming, including, for example, milk value, or whether a specific lot of milk should be routed for use in a specific dairy product, such as cheese, yogurt, cream, or drinking milk. Measurements and understanding of these components also can be important to herd management, for example as an indicator of herd health and/or disease. For example, a level of one or more of these components of milk can be used in early recognition and intervention, which can stop the spread of disease in a herd and reduce losses to the herd and milk product.

In the milk industry, those of ordinary skill in the art thus understand that the various milk models (and hence, resulting classifications) may be based on homogenized milk measurements, and therefore, corrections to measurements of non-homogenized milk based on the homogenized-milk models, will lead to mischaracterization and/or misclassification of milk samples, and to the associated stored milk.

The present disclosure thus provides for methods, systems, and apparatus for measuring a component (e.g., fat) of non-homogenized samples, e.g., milk, and computing a milk fat measurement based on standard homogenized milk fat models, and generating a correction factor that may be applied to the milk fat measurement. The correction factor is based on a particle size, which may include a mean particle size, where particle size is understood to be a diameter of particles of the component being measured. As disclosed, a mean particle size may be selected such that a certain proportion of particles of the component, e.g., milkfat, are less than the selected particle size.

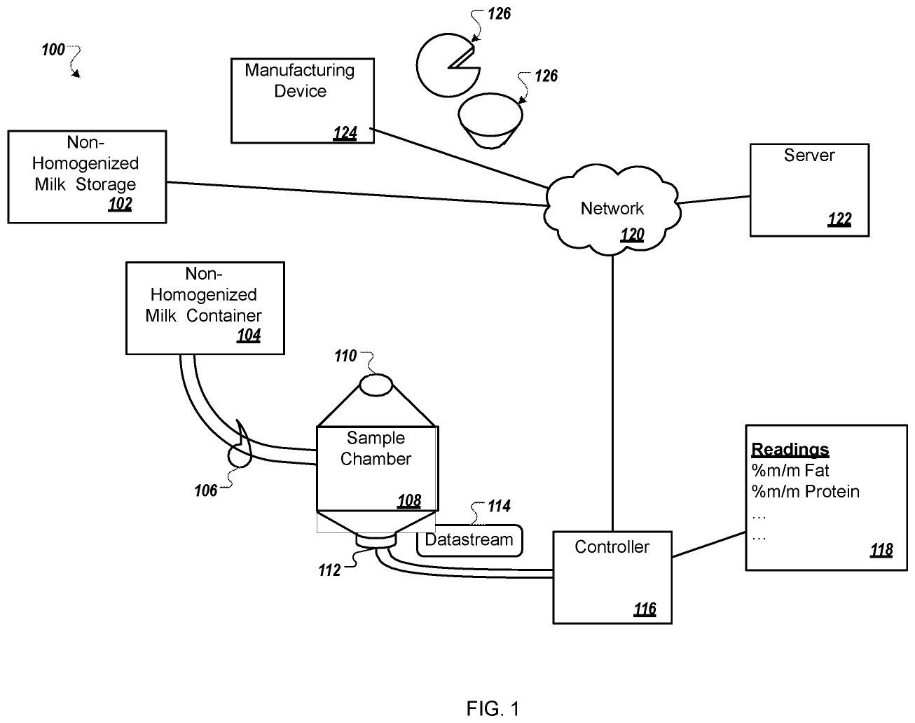

FIG. 1 is a conceptual diagram of an example system 100 that can be used for determining a measure of a component of a fluid sample. In the depicted system 100, a storage 102 holds a quantity of non-homogenized milk, some of which is loaded into a container 104, although those of ordinary skill will understand that the present disclosure is not limited to a particular sample type. A sample 106 of the non-homogenized milk is extracted from the container 104 and loaded into a sample chamber 108. With the sample 106 loaded into the sample chamber 108, an energy source 110 energizes and emits infrared energy into the sample 106. A sensing element 112 is positioned to be exposed to the resulting (e.g., infrared) energy that is received from the sample, and the sensing element 112 is configured to transmit a corresponding datastream 114 to a controller 116. From the datastream 114, the controller 116 determines readings 118 of certain contents, components, and/or constituents of the sample 106. The readings 118 can be transmitted over a wired or wireless network 120 to a server 112, to a manufacturing device 124, or to other destinations/devices. The readings can be stored to computer memory, used to initiate or modify a manufacturing process to make food products 126 (e.g., cheese, yogurt,) or for other purposes.

In some embodiments, the storage 102 (e.g., non-homogenized milk storage) can include various large fluid containers, including stationary tanks, railroad cars, truck trailers, etc., that are configured to hold fluids. In some embodiments, the storage is configured to hold non-homogenized milk. A container 104 of the non-homogenized milk can be drawn from the storage 102 and a sample of the non-homogenized milk can be extracted. For example, a dewar can be filled from the storage 102, and a pipette can be loaded with the non-homogenized milk.

In some embodiments, without homogenizing the non-homogenized milk, the sample 106 can be loaded into the sample chamber 108 for irradiation by the energy source 110. For example, a single device may include the energy source 110, the sample chamber 108, the sensor 112, and the controller. A user may load the sample 106 into the device using the pipette and enter instructions in an interface panel of the device to command the device to measure one or more components of the non-homogenized milk.

Using other technologies, to measure the multiple components of solutions such as milk, the solution must first be treated by a process known as homogenization. Homogenization of a solution is the process of breaking down the particle size of the components in the solution to a more homogeneous mixture of smaller, similar particle sizes. For example, the fat component found in milk is composed of a heterogeneous mixture of fat particles that include a wide range of particle sizes. This wide size range of particles makes it difficult for a measurement instrument, typically a spectrometer, to accurately detect and provide a measurement value for the fat component. In the system 100 depicted in FIG. 1, homogenization is not required, which provides advantages over methods and systems that require homogenization in order to measure components within a solution.

In some embodiments, the readings 118 contain data generated from the sensing. For example, the readings 118 can include data listing the percentage, by mass, of various components of a solution (e.g., a non-homogenized solution). This data is sometimes expressed as “% m/m.” However, it will be understood that other formats can be used to describe the components of a solution.

In some embodiments, the network 120 includes data networking hardware and software used to allow transmission of data messages between various components. In some embodiments, the network 120 may include the Internet and/or one or more other networks, including private networks. In some embodiments, the server 122 includes one or more real or virtual computing devices that can receive the readings 118 and act upon the readings (e.g., storing the readings to disk, using the readings in an analysis process). In some embodiments, the manufacturing device 124 includes one or more machines and/or their controllers that use the non-homogenized milk from the storage 102 in the manufacture of one or more products. For example, some manufacturing devices can route the non-homogenized milk (e.g., through pipes) so that the non-homogenized milk can be used for the manufacture of cheese, cream, or other products derivable from non-homogenized milk.

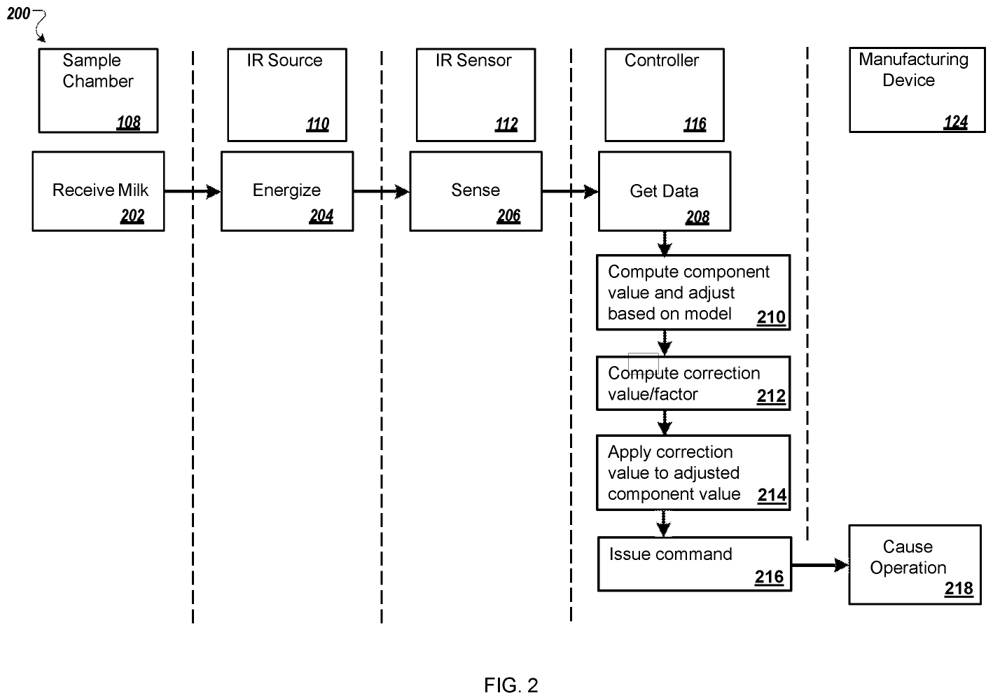

FIG. 2 is a flowchart of an example process 200 used to perform operations on a fluid. In some embodiments, the process 200 can be performed by, for example, elements of the system 100, and as such will be described with reference to some of those elements. In some embodiments, one or more systems other than those described herein can be used to perform the process 200 or other process on a fluid.

In some embodiments, the sample chamber 108 receives a sample of non-homogenized solution containing one or more components 202. For example, a human technician can connect a hose from the container 104 and transmit the sample 106 of non-homogenized milk from the container to the sample chamber 108.

In some embodiments, the infrared energy source 110 is energized and emits infrared energy into the sample 204 in a given, selected wavenumber range (e.g., any of the exemplary wavenumbers or ranges of wavenumbers described herein). In some embodiments, the human technician can interact with interface elements (e.g., buttons, dials, read-outs) to begin the sensing process. In response, the controller 116 can send a command to the infrared energy source 110 to energize, and the infrared energy source 110 energizes. The wavenumber range of the emitted energy may be selected based on, for example, the type of sample (e.g., non-homogenized milk), and/or the component (e.g., fat content) of the sample to be measured. In some embodiments, the wavenumber range of the emitted energy may include a wide range of wavenumbers, for example, the entire MIR range and/or some other range that may be user-defined and/or established by the instrument itself that is responsible for the emission.

In some embodiments, the infrared sensor 112, comprising a sensing element positioned to be exposed to a portion of the emitted infrared energy that has been emitted into the sample, generates a datastream based on the exposure of the sensing element to the emitted infrared energy 206. For example, the infrared sensor 112 can include elements that, when exposed to infrared energy, generate electrical signals that are proportional (e.g., in the domain of amplitude and/or frequency) to the received energy. The infrared sensor 112 can transmit these signals as analog data or convert these signals into digital signals for transmission to the controller 116. The controller 116 receives the datastream 208. The data and/or datastream can be understood as and/or represented as an emission spectrum. In some embodiments, the emission spectrum can be converted, e.g., through Fourier transform, to an absorption spectrum. In some embodiments, the “measured” (e.g., unadjusted, uncorrected) components of a fluid can be computed using the determined absorption spectrum. In some embodiments, the “measured” (e.g., unadjusted, uncorrected) components of a fluid can be computed using the determined emission spectrum.

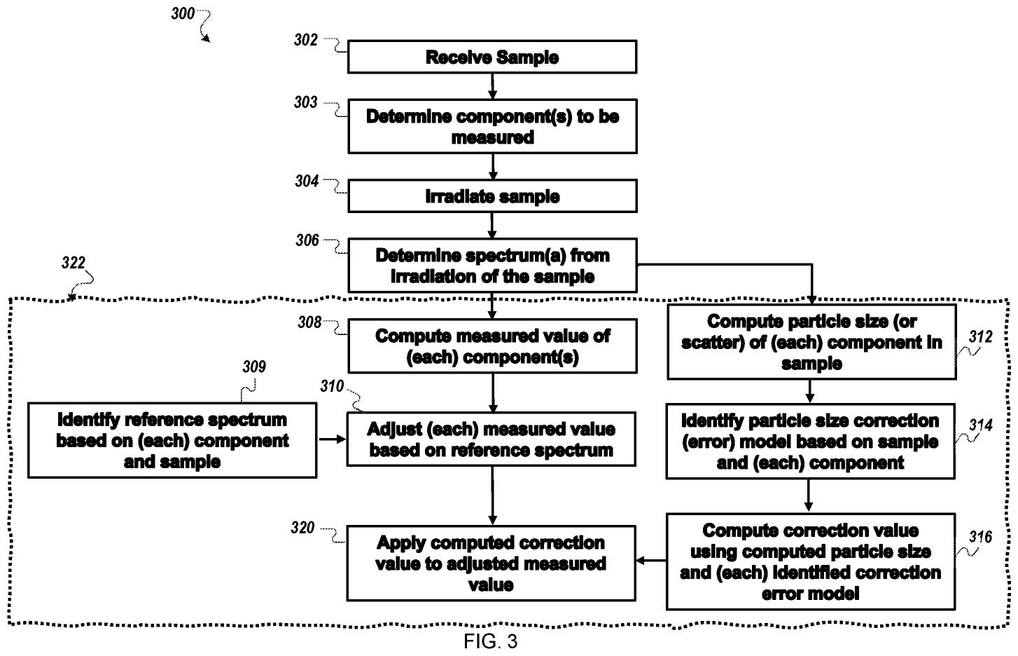

In some embodiments, the controller 116 computes the value of the measured component based on the received spectral data (e.g., the determined absorption spectrum and/or the determine emission spectrum), and adjusts the value based on at least one reference model (e.g., based on homogenized samples) 210. For example, the measured value may be adjusted based on a comparison of the measured/determined spectrum (e.g., absorption spectrum) and the reference spectrum. The adjusted value may be computed using a regression such as a partial least square regression, to determine a linear relationship between the determined/measured spectrum of the measured component and an associated model of the component. Those of ordinary skill will understand that the comparison of the two spectra (e.g., measured spectrum and reference spectrum) can be performed in a variety of different manners as known in the art, including but not limited to multiple types of regression techniques (e.g., least squares, ridge, polynomial, Bayesian, logistic, and Lasso), and the present disclosure is not limited to such techniques. An example process for determining readings is described later with respect to FIG. 3.

In some embodiments, a correction value is computed 212 from the determined/measured spectral data. The correction value is then applied to the adjusted value of the measured component 214. In some embodiments, the controller 116 optionally issues a command 216. For example, the controller may compute that the non-homogenized milk sample has a particular % m/m fat content. This value is compared with a ruleset that specifies different uses based on different % m/m values. For example, the controller may generate a command based on the previous determination. In such cases, the controller determines that the non-homogenized milk sample includes a % m/m fat content that match a rule from the ruleset indicating use of the non-homogenized milk for the manufacture of drinking milk. As such, the controller 116 generates a command to transfer the non-homogenized milk to a manufacturing device 124 that manufactures drinking milk.

In some embodiments, the manufacturing device 124 causes an operation 218. For example, the manufacturing device 124 initiates a manufacturing process to manufacture drinking milk from the non-homogenized milk.

Those of ordinary skill will understand that the embodiment as depicted in FIG. 2 is a non-limiting example of the methods and systems described herein. Accordingly, aspects shown in FIG. 2 may be combined, rearranged, or eliminated entirely, depending on the embodiment, without departing from the scope of the disclosed method and systems. For example, those of ordinary skill will understand that the measured value and adjusted value may be computed in a single computation and need not be performed separately as depicted. Accordingly, the present disclosure includes determining an adjusted value of the component based on a comparison of the determined/measured spectrum and a reference spectrum. In such an embodiment, a measured value of the component may not be separately computed.

FIG. 3 is a flowchart of a non-limiting exemplary process 300 for determining measurements of components in fluid samples. The process 300 can be performed, for example, using elements of the system 100, and as such will be described with reference to some of those elements. In some embodiments, one or more additional systems not described herein can be used to perform the process 300 on a fluid. In some cases, the process 300 can be used as a part of the process 200 as depicted in FIG. 2.

Referring to FIG. 3, a sample comprising a non-homogenized solution is received 302. For example, the sample chamber 108 can receive a small sample of milk that is used to represent a larger volume of milk. The sample can be tested to identify one or more properties of the milk. In such cases, one or more devices may perform the sensing of the process 300, as opposed to a human operator performing complex calculations. In some embodiments, the one or more devices that perform the sensing in the process for 300 can be configured to generate and sense natural phenomena that are tied to this process, such as the generation of infrared energy.

In some cases, the sample is either an animal dairy product or a non-animal dairy product. For example, the sample can be a dairy product such as raw milk, milk, cream, ice cream, yogurt, cheese, or a combination of these. This may be useful, for example, in the testing of dairy products as part of a manufacturing process. For example, a device incorporating the technology described here may be installed in a manufacturing facility to test food products that are manufactured in the facility. The testing can thus be performed on, e.g., milk from various sources. In some cases, the sample can include milk from cow, sheep, camel, buffalo, goat, and/or human sources. In some cases, the sample can include, without limitation, nut-milk, baby formula, meal replacement drinks, non-dairy milk, plant-based milk (e.g., almond, oat, soy, rice, nut, peanut, coconut, sesame, cashew, and hemp) and liquid livestock feeds. In some cases, the sample is a solution containing one or more components selected from at least one of fat, protein, total protein, true protein, lactose, non-protein nitrogen, urea, solids, or non-fat solids, or any combinations thereof. Additional components can include vitamin fortifications, and non-dairy fats. In some embodiments, a component of the solution is determined (e.g., identified) to be measured 303, although such determination can occur at other times in the depicted process. In a non-limiting example, a component (e.g., fat content) to be measured can be determined after irradiating the sample.

In some cases, and based on the component to be measured, the sample is heated. In some embodiments, the sample can be heated to between about 2° C. to about 42° C. (e.g., between about 2° C. to about 40° C., between about 2° C. to about 35° C., between about 2° C. to about 30° C., between about 2° C. to about 25° C., between about 2° C. to about 20° C., between about 2° C. to about 15° C., between about 2° C. to about 10° C., between about 2° C. to about 5° C., between about 5° C. to about 42° C., between about 5° C. to about 40° C., between about 5° C. to about 35° C., between about 5° C. to about 30° C., between about 5° C. to about 25° C., between about 5° C. to about 20° C., between about 5° C. to about 15° C., between about 5° C. to about 10° C., between about 10° C. to about 42° C., between about 10° C. to about 40° C., between about 10° C. to about 35° C., between about 10° C. to about 30° C., between about 10° C. to about 25° C., between about 10° C. to about 20° C., between about 10° C. to about 15° C., between about 15° C. to about 42° C., between about 15° C. to about 40° C., between about 15° C. to about 35° C., between about 15° C. to about 30° C., between about 15° C. to about 25° C., between about 15° C. to about 20° C., between about 20° C. to about 42° C., between about 20° C. to about 40° C., between about 20° C. to about 35° C., between about 20° C. to about 30° C., between about 20° C. to about 25° C., between about 25° C. to about 40° C., between about 25° C. to about 40° C., between about 25° C. to about 35° C., between about 25° C. to about 30° C., between about 30° C. to about 42° C., between about 30° C. to about 40° C., between about 30° C. to about 35° C., between about 35° C. to about 42° C., between about 35° C. to about 40° C., or between about 40° C. to about 42° C.). In some embodiments, the sample can be heated to between about 35° C. to about 42° C. degrees. In some embodiments, the sample can be heated to about 40° C. In some embodiments, heating can ensure that a particular viscosity is achieved, and/or that all samples are tested under substantially consistent conditions. In some embodiments, for example, cream milk products, heating may be desired for increased viscosity; however, in other embodiments using other sample types, for example, dairy milk, heating to, e.g., 40° C. will cause the fat to dissolve, thereby making heating desirable in some cases.

As shown in the exemplary example in FIG. 3, the sample is irradiated 304. For example, the IR source 110 can be energized to cause the IR source 110 to emit IR energy into the sample. In doing so, the IR energy is altered based on the interaction with the sample. In some cases, for example, in the case of Fourier-transform infrared spectroscopy (FTIR), the IR can emit at a range of wavenumbers that can be, e.g., in a range of about 400 cm⁻¹ to about 4000 cm⁻¹ (e.g., about 400 cm⁻¹ to about 3500 cm⁻¹, about 400 cm⁻¹ to about 3000 cm⁻¹, about 400 cm⁻¹ to about 2500 cm⁻¹, about 400 cm⁻¹ to about 2000 cm⁻¹, about 400 cm⁻¹ to about 1500 cm⁻¹, about 400 cm⁻¹ to about 1000 cm⁻¹, about 400 cm⁻¹ to about 900 cm⁻¹, about 400 cm⁻¹ to about 800 cm⁻¹, about 400 cm⁻¹ to about 700 cm⁻¹, about 400 cm⁻¹ to about 600 cm⁻¹, about 400 cm⁻¹ to about 500 cm⁻¹, about 500 cm⁻¹ to about 4000 cm⁻¹, about 500 cm⁻¹ to about 3500 cm⁻¹, about 500 cm⁻¹ to about 3000 cm⁻¹, about 500 cm⁻¹ to about 2500 cm⁻¹, about 500 cm⁻¹ to about 2000 cm⁻¹, about 500 cm⁻¹ to about 1500 cm⁻¹, about 500 cm⁻¹ to about 1000 cm⁻¹, about 500 cm⁻¹ to about 900 cm⁻¹, about 500 cm⁻¹ to about 800 cm⁻¹, about 500 cm⁻¹ to about 700 cm⁻¹, about 500 cm⁻¹ to about 600 cm⁻¹, about 600 cm⁻¹ to about 4000 cm⁻¹, about 600 cm⁻¹ to about 3500 cm⁻¹, about 600 cm⁻¹ to about 3000 cm⁻¹, about 600 cm⁻¹ to about 2500 cm⁻¹, about 600 cm⁻¹ to about 2000 cm⁻¹, about 600 cm⁻¹ to about 1500 cm⁻¹, about 600 cm⁻¹ to about 1000 cm⁻¹, about 600 cm⁻¹ to about 900 cm⁻¹, about 600 cm⁻¹ to about 800 cm⁻¹, about 600 cm⁻¹ to about 700 cm⁻¹, about 700 cm⁻¹ to about 4000 cm⁻¹, about 700 cm⁻¹ to about 3500 cm⁻¹, about 700 cm⁻¹ to about 3000 cm⁻¹, about 700 cm⁻¹ to about 2500 cm⁻¹, about 700 cm⁻¹ to about 2000 cm⁻¹, about 700 cm⁻¹ to about 1500 cm⁻¹, about 700 cm⁻¹ to about 1000 cm⁻¹, about 700 cm⁻¹ to about 900 cm⁻¹, about 700 cm⁻¹ to about 800 cm⁻¹, about 800 cm⁻¹ to about 4000 cm⁻¹, about 800 cm⁻¹ to about 3500 cm⁻¹, about 800 cm⁻¹ to about 3000 cm⁻¹, about 800 cm⁻¹ to about 2500 cm⁻¹, about 800 cm⁻¹ to about 2000 cm⁻¹, about 800 cm⁻¹ to about 1500 cm⁻¹, about 800 cm⁻¹ to about 1000 cm⁻¹, about 800 cm⁻¹ to about 900 cm⁻¹, about 900 cm⁻¹ to about 4000 cm⁻¹, about 900 cm⁻¹ to about 3500 cm⁻¹, about 900 cm⁻¹ to about 3000 cm⁻¹, about 900 cm⁻¹ to about 2500 cm⁻¹, about 900 cm⁻¹ to about 2000 cm⁻¹, about 900 cm⁻¹ to about 1500 cm⁻¹, about 900 cm⁻¹ to about 1000 cm⁻¹, about 1000 cm⁻¹ to about 4000 cm⁻¹, about 1000 cm⁻¹ to about 3500 cm⁻¹, about 1000 cm⁻¹ to about 3000 cm⁻¹, about 1000 cm⁻¹ to about 2500 cm⁻¹, about 1000 cm⁻¹ to about 2000 cm⁻¹, about 1000 cm⁻¹ to about 1500 cm⁻¹, about 1500 cm⁻¹ to about 4000 cm⁻¹, about 1500 cm⁻¹ to about 3500 cm⁻¹, about 1500 cm⁻¹ to about 3000 cm⁻¹, about 1500 cm⁻¹ to about 2500 cm⁻¹, about 1500 cm⁻¹ to about 2000 cm⁻¹, about 2000 cm⁻¹ to about 4000 cm⁻¹, about 2000 cm⁻¹ to about 3500 cm⁻¹, about 2000 cm⁻¹ to about 3000 cm⁻¹, about 2000 cm⁻¹ to about 2500 cm⁻¹, about 2500 cm⁻¹ to about 4000 cm⁻¹, about 2500 cm⁻¹ to about 3500 cm⁻¹, about 2500 cm⁻¹ to about 3000 cm⁻¹, about 3000 cm⁻¹ to about 4000 cm⁻¹, about 3000 cm⁻¹ to about 3500 cm⁻¹, or about 3500 cm⁻¹ to about 4000 cm⁻¹).

In some embodiments, the selected wavenumber for irradiation is dependent on the sample. For example, wavenumbers in the FTIR range (e.g., any of the wavenumbers or ranges of wavenumbers described herein) are known to be useful for analyzing milk.

In some embodiments, the output of the sensor 112 can thus comprise one or more spectrum corresponding to the emitted wavenumbers as modified by the sample. In some embodiments, one or more spectrum such as an infrared emission spectrum and/or an infrared absorption spectrum may be determined from the irradiation of the sample 306. For example, the controller 116 can access a computer memory location in which one more spectrum values are stored.

In some embodiments, the one or more spectrum and/or data related thereto are processed to generate a measured value of an amount of a component of the sample (e.g., fat content). In some embodiments, generation of a measured value of an amount of the component includes the use of a reference model/spectrum related to the component (e.g., fat content) for samples of the same type (e.g., milk fat model for heavy cream) 308, 310. In some embodiments, the reference spectrum may be based on industry standards for the component (e.g., fat content). In some embodiments, the reference model includes a model for homogenized samples (e.g., the reference model is a model for homogenized milk where the sample being test is non-homogenized milk) where the same component (e.g., fat content) is compared between the homogenize sample (e.g., homogenized milk) and non-homogenized sample (e.g., non-homogenized milk).

In some cases, the first component comprises one or more of a fat, a protein, total protein, true protein, lactose, a non-protein nitrogen (e.g., urea), solids, or non-fat solids, or any combinations thereof. In some cases, the first component is fat. As will be understood, this list of possible components includes components found in milk. When a different fluid is processed, one or more different components found in those fluids may be used in the methods and systems described herein.

In some embodiments where the sample is non-homogenized milk, the representation of the component (e.g., the measured value of the component) may be a measurement of fat content, where the first representation or measured value is based on an absorption spectrum (derived as described herein) 308. The measured value of the component may be adjusted by a comparison or a fitting of the absorption spectrum to one or more standard, industry-accepted, or other mathematical (“reference”) spectrum/models of absorption spectrum for fat content (for that type of milk) in a homogenized solution 310. Those of ordinary skill in the art will thus understand that the adjusted value may be based on a comparison of the measured spectrum and one or more mathematical models 310 such as Fat A, Fat B, Fat C, Fat D, or a Fat Partial-Least-Squares (“PLS”) model. Accordingly, in some embodiments, the process 300 includes generating the first representation/measured value (e.g., unadjusted/uncorrected) of the component (e.g., fat) with one or more numerical values based on the relationship between the measured spectrum and the spectrum of a selected reference model/spectrum 309. In some embodiments, a Fat A model is based on wavenumbers from about 1740 cm⁻¹ to about 1760 cm⁻¹ (or any of the subranges therein). In some embodiments, a Fat B model is based on wavenumbers from about 2834 cm⁻¹ to about 2874 cm⁻¹ (or any of the subranges therein). In some embodiments, a Fat C model is based on wavenumbers from about 1440 cm⁻¹ to about 1480 cm⁻¹ (or any of the subranges therein). In some embodiments, a Fat D model is based on wavenumbers from about 1137 cm⁻¹ to about 1177 cm⁻¹ (or any of the subranges therein). In some embodiments, a Fat PLS model includes wavenumbers that range across the complete FTIR spectrum.

In some embodiments, the designation of and/or selection of an appropriate reference model/spectrum 309 can be based on the sample type and characteristics, the component being measured, and/or the type of data being analyzed (e.g., without limitation, absorption spectrum and emission spectrum). In some embodiments, the sample is milk (e.g., non-homogenized milk) and the component is fat. In some embodiments, this disclosure features methods and systems for measuring a component, wherein measurement of the component comprises measuring the content and/or amount of components of milk, such as, e.g., protein, total protein, true protein, lactose, urea, and/or other non-protein nitrogen. In such cases, the process includes, for example, a library of reference models/spectra 309 can be selected according to the component to be measured, e.g., for fat content, protein (total, true) content, a lactose content, urea, other non-protein nitrogen content, or any combinations thereof. In some embodiments, a reference model used for measuring protein in a non-homogenized solution includes a protein model that includes and/or is based on wavenumbers from about 1531 cm⁻¹ to about 1551 cm⁻¹ (or any of the subranges therein). In some embodiments, a reference model used for measuring total protein in a non-homogenized solution includes and/or is based on a total protein model that is a PLS model using all the infrared spectrum. In some embodiments, a reference model used for measuring true protein in a non-homogenized solution includes and/or is based on a true protein model that is a PLS model using the infrared spectrum. In some embodiments, a reference model used for measuring lactose in a non-homogenized solution includes a lactose model that includes and/or is based on wavenumbers from about 1038 cm⁻¹ to about 1058 cm⁻¹ (or any of the subranges therein). In some embodiments, a reference model used for measuring solids in a non-homogenized solution includes a solids model that includes the addition of fat, protein, lactose, and, minerals. In some embodiments, a reference model used for measuring solids, non-fat (SNF) in a non-homogenized solution includes a model that includes the addition of protein, lactose, and, minerals.

In some embodiments, the comparison of the measured (e.g., absorption) spectrum to the reference spectrum may allow for a computation of the amount (e.g., percentage or absolute value) of the component (e.g., fat content) by determining a fat content from the measured spectrum 308, and adjusting it based on the comparison of the two spectra 310. For example, in certain embodiments, the comparison of measured and reference spectra may include a regression such as, e.g., a least squares regression or linear least squares regression, that results in a correction to the measured fat content of the form: (adjusted) Fat Content=m*(measured Fat Content)+b, where m and b are the slope and y-intercept resulting from the linear regression. Those of ordinary skill will understand that the present disclosure is not limited to the method of regression analysis and/or the specific method of adjusting or compensating the measured component value based on the model, and that other techniques may be used.

In the FIG. 3 embodiment, an adjustment to the measured value is made 310, however it can be understood that the processes shown in 308 and 310 may be combined in a single process, such that a computation of a measured value and adjusted value may be performed concurrently (or sequentially) through the comparison of the measured spectrum with the reference spectrum 308, 310.

The inventors have surprisingly found that, for the different reference models of a given component, a linear relationship can be determined between particle size and measurement error. For example, when the component is fat, for a given fat reference model/spectrum (e.g., Fat A), the inventors have found that a relationship between the particle size of the measured component and a given measurement error can be computed and/or determined and used to correct the representation (e.g., measured value) of the component (e.g., fat content) being measured. In some embodiments, the relationship is a linear relationship. In some embodiments, the adjustment value is thus based on the particle size of the component and a linear relationship to measurement error (e.g., difference between homogenized and non-homogenized measurements of the component), with the particle size and error model being related to and/or associated with the component being measured 314.

In some embodiments, particle size of a non-homogenized sample is determined, wherein the particle size data can be used to correct (e.g., via the determination of a correction factor) the adjusted value (e.g., the adjusted measured value) 320 and remove substantial error associated with applying non-homogenized measurements to homogenized reference models/spectra. In certain embodiments, the particle size information includes D₉₀, known in the art as the particle size for which 90% of the particles are below/less than. In some embodiments, other measures of particle size may be used. In some embodiments, the particle size information is based on the mean particle size, and more specifically, the mean diameter of the particles of the component being measured in at least a portion of the sample. In some embodiments, particle size (e.g., without limitation, mean particle size and D90) is also computed based on the measured spectrum (e.g., without limitation, emission spectra and/or absorption spectra) 312.

In some cases, the particle size is a measure of a diameter. For example, fat particles in non-homogenized milk can be up to 20 μm. Non-limiting examples of mean particle sizes include less than 3 μm, less than 2 μm, or less than 1 μm. In some embodiments, mean particle size is about 0.1 μm to about 3 μm (e.g., about 0.1 M to about 2 μM, about 0.1 μM to about 1 μM, about 0.1 μM to about 0.5 μMm, about 0.1 μM to about 0.25 μM, 0.25 μM to about 3 μM, about 0.25 μM to about 2 μM, about 0.25 μM to about 1 μM, about 0.25 μM to about 0.5 μMm, about 0.5 μM to about 3 μM, about 0.5 μM to about 2 μM, about 0.5 μM to about 1 μM, about 1 μM to about 3 μM, about 1 μM to about 2 μM, or about 2 μM to about 3 μM).

In some embodiments, the methods provided herein include using a selected particle size. In some embodiments, the selected particle size is predetermined. For example, the selected particle size is determined prior to commencing the methods described herein. In some embodiments, the particle size is selected after the methods described herein have been performed on the solution. In some embodiments, the methods provided herein include determining the particle size, wherein the determining step occurs at any point during the method. In some embodiments, the selected particle size is associated with and/or based on the component of the sample. In some embodiments, the selected particle size is determined based on a predetermined proportion of particles of the component in at least a portion of the sample having a mean diameter less than the selected particle size. In some embodiments, a predetermined proportion of particles refers to a proportion of the solution. In some embodiments, a predetermined proportion of particles includes a portion between about 20% and about 90% (e.g., about 20% to about 80%, about 20% to about 70%, about 20% to about 60%, about 20% to about 50%, about 20% to about 40%, about 20% to about 30%, about 30% to about 90%, about 30% to about 80%, about 30% to about 70%, about 30% to about 60%, about 30% to about 50%, about 30% to about 40%, about 40% to about 90%, about 40% to about 80%, about 40% to about 70%, about 40% to about 60%, about 40% to about 50%, about 50% to about 90%, about 50% to about 80%, about 50% to about 70%, about 50% to about 60%, about 60% to about 90%, about 60% to about 80%, about 60% to about 70%, about 70% to about 90%, about 70% to about 80%, or about 80% to about 90%) of the sample. In some embodiments, the predetermined proportion is 90%.

In some embodiments, a particle scatters infrared energy based on size distribution, size, and composition of the particles. In some embodiments, scatter created by a particle is detected in the measured spectrum. In some embodiments, particle scatter is measured at wavenumbers ranging from about 3700 cm⁻¹ to about 3800 cm⁻¹. In some embodiments, particle scatter is measured at wavenumbers ranging from about 3740 cm⁻¹ to about 3760 cm⁻¹ (or any of the subranges therein).

In some embodiments, the methods provided herein can be used to compute an amount of a component, a preliminary value, a representation, or a measured value of a non-homogenized sample 308. The measured value can correspond to and/or be computed based on a measurement taken as if the non-homogenized sample 106 was actually a homogenized sample 308. For example, an analysis traditionally used for homogenized milk may be applied to non-homogenized milk.

In some embodiments, an adjustment factor can be applied to the measured value based on a comparison to a reference model for the particular sample, and for a homogenized sample of that type 310. In some embodiments, the one or more determined spectrum are used to generate an adjustment factor. In some embodiments where processing the one or more determined spectrum is used to generate an adjustment factor, the method also includes determining a reference spectrum associated with the component and comparing the one or more determined spectrum to the reference spectrum to generate the adjustment factor. In some embodiments, wherein comparing the one or more determined spectrum to the reference spectrum includes performing a linear least squares regression. In some embodiments, processing the one or more determined spectrum to generate an adjustment factor further includes determining a reference spectrum associated with the component and comparing the determined spectrum to the reference spectrum to generate the adjustment factor where the reference spectrum is based on one or more mathematical models selected from Fat A model, Fat B model, Fat C model, Fat D model, or Fat PLS model. In some embodiments, the adjustment factor improves the accuracy of a measured value for when the sample is not homogenized. The adjusted value can then be corrected using particle size 320.

In some cases, a correction factor can be created based on a measurement of particle size or particle scatter of the components within the sample 106, 312. For example, the particle size or particle scatter may be determined using an absorption spectrum that is determined or computed using the sensed infrared energy emitted from the irradiated sample. In embodiments where the correction factor is based on particle size, the correction factor can be created based on, for example, without limitation: median size of the particles, size of at least half of the particles, and a mean particle size that falls within 90% of the particles (i.e., D₉₀). In some embodiments, the correction factor is based on a relationship between the error measurement and the selected particle size, where the relationship corresponds to and/or is associated with a reference model associated with the component. In some embodiments, the correction factor is based on a relationship between the error measurement and particle size for at least one of: Fat A model, Fat B model, Fat C model, Fat D model, or Fat PLS model. In some embodiments, the correction factor is based on D₉₀ or any of the proportional subranges described herein and particle size (e.g., any of the exemplary particle sizes described herein). In some embodiments, the correction factor is based on particle scatter (e.g., scatter caused by particles (e.g., fat particles) as detected from the measured spectrum). In some embodiments, particle scatter is used in place of particle size in creating the correction factor. In some embodiments, particle size and particle scatter are both used, at least in part, in creating the correction factor.

In some embodiments, the adjusted value is modified using the correction factor to generate a corrected value of the component. In such cases, the corrected value of the component is an indication of an amount of the component present in the sample.

As shown in FIG. 3, the disclosed methods and systems can be repeated for different components of a sample. In such cases, certain aspects of the methods and systems provided herein can be repeated for two or more different components of the sample. In some embodiments, a single measured spectrum may be used for two or more components. In some embodiments when a single measured spectrum is used for measuring two or more components, the disclosed methods include selection of different reference spectra 309 and different error models 314 based on the components being measured. In some embodiments, the process as described in FIG. 3 is repeated for different components of the same sample.

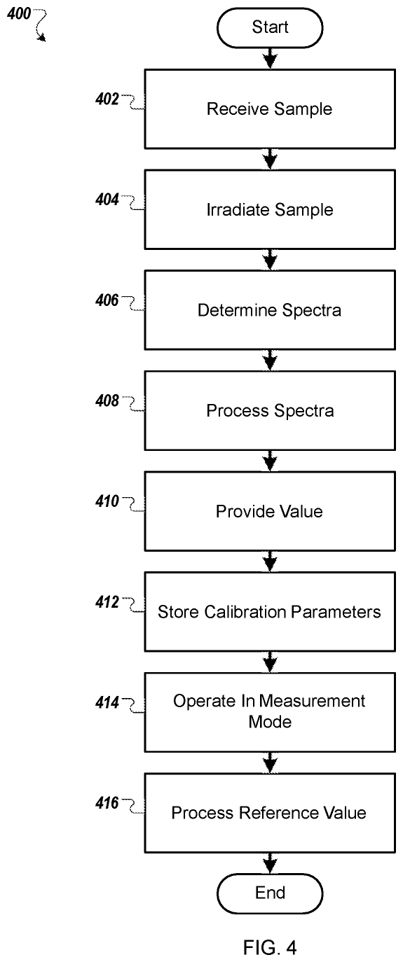

FIG. 4 is a flowchart of an example process 400 for calibration. The process 400 can be performed, for example, elements of the system 100, and as such will be described with reference to some of those elements. However, it will be understood that other systems can be used to perform the process 400 on a fluid or solution. In some cases, the process 400 can be used as a part of the process 200. For example, after computing and applying the correction factor as described herein, a further calibration adjustment may be made.

As illustrated in FIG. 4 sample comprising a reference solution containing one or more reference particles is received 402. For example, a sample of fluid having known properties can be sources and used for the calibration. A sample of either real milk having a known compositional profile, or a milk analog synthesized from other fluids may be used, for example. This sample may have known quantities of components that are generally in similar proportions to the component profile of real milk that will be tested later using the same equipment.

The sample is irradiated with infrared energy 404 and one or more spectrum from infrared absorption spectrum and infrared emission spectrum are determined based on the one or more reference particles 406. For example, the operator of the machine may use interface elements (e.g., buttons) to instruct the device to begin the calibration process. In response, the device can energize and collect the spectrum data.

The one or more spectrum from the reference particles are processed to generate a representation of an amount of one or more reference particles 408. For example, based on an uncalibrated reading, the device can generate an initial value for some component of the fluid. In the case of a fat measurement, an initial % m/m value can be generated.

A value of one or more reference particles of the reference solution is provided 410. For example, the operator can enter into the device the known quantity of fat, in % m/m, of the sample.

Calibration parameters generated from the one or more reference particles are stored 414. Based on the difference between the initial % m/m value and the entered % m/m values, calibration parameters representing the difference may be calculated. In general, the calculation parameters are a set of parameters that, when applied to the initial % m/m values, generate the known % m/m value.

In one example, the initial % m/m values and the known % m/m values may each use a linear model. In this example, the calibration parameters are parameters that mathematically define the difference between the slopes of the two lines, so that a particular value on one line (e.g., an initial % m/m) can be associated with a single value on the other line (e.g., a corresponding known % m/m value). In some cases, this relationship can include a slope and intercept values that define, for example, where the two lines intersect and the difference between their slopes.

A reference value according to the equation is processed from the calibration parameters 416. For example, when used in full production, measurements of a component to which the correction value has been applied can be further adjusted based on these stored calibration values.

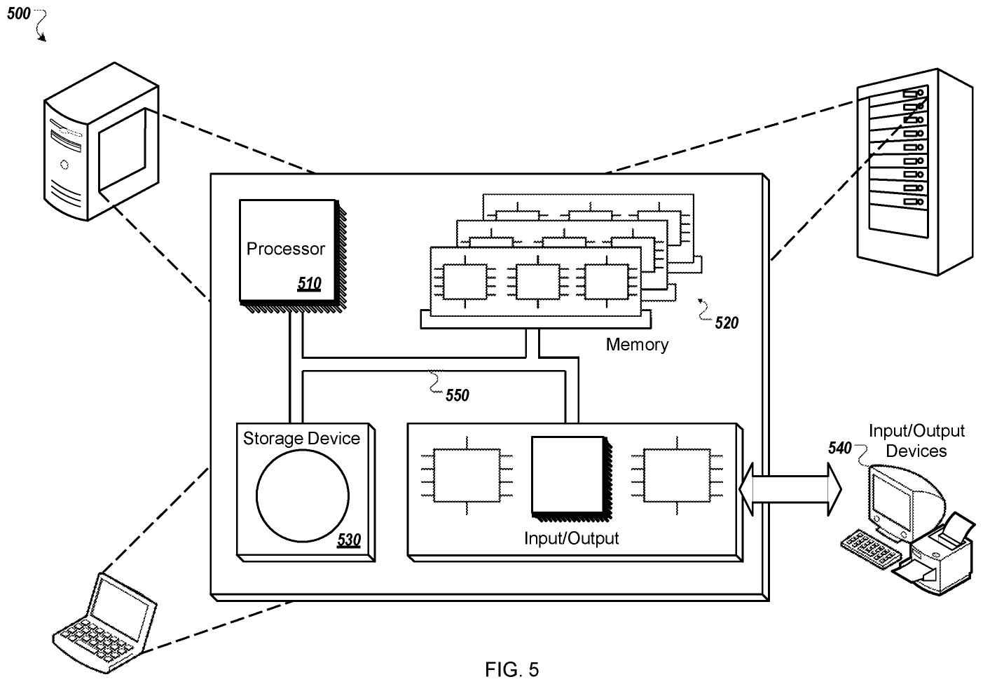

FIG. 5 is a schematic diagram that shows a non-limiting example of a computing system 500. The computing system 500 can be used for some or all of the operations described previously, according to some implementations. The computing system 500 includes a processor 510, a memory 520, a storage device 530, and an input/output device 540. Each of the processor 510, the memory 520, the storage device 530, and the input/output device 540 are interconnected using a system bus 550. The processor 510 is capable of processing instructions for execution within the computing system 500. In some implementations, the processor 510 is a single-threaded processor. In some implementations, the processor 510 is a multi-threaded processor. The processor 510 is capable of processing instructions stored in the memory 520 or on the storage device 530 to display graphical information for a user interface on the input/output device 540.

The memory 520 stores information within the computing system 500. In some implementations, the memory 520 is a computer-readable medium. In some implementations, the memory 520 is a volatile memory unit. In some implementations, the memory 520 is a non-volatile memory unit.

The storage device 530 is capable of providing mass storage for the computing system 500. In some implementations, the storage device 530 is a computer-readable medium. In various different implementations, the storage device 530 may be a floppy disk device, a hard disk device, an optical disk device, or a tape device.

The input/output device 540 provides input/output operations for the computing system 500. In some implementations, the input/output device 540 includes a keyboard and/or pointing device. In some implementations, the input/output device 540 includes a display unit for displaying graphical user interfaces.

In some embodiments, features described herein can be implemented in digital electronic circuitry, or in computer hardware, firmware, software, or in combinations of them. The apparatus can be implemented in a computer program product tangibly embodied in an information carrier, e.g., in a machine-readable storage device, for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment. Accordingly, as used herein, a “computer” may be understood to be a device comprising at least one microprocessor (e.g., a desktop, a laptop, a tablet, and a phone), where such a device can be configured to perform at least some of the functionality described herein. A “microprocessor” may also be referred to as a “processor.”

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM (erasable programmable read-only memory), EEPROM (electrically erasable programmable read-only memory), and flash memory devices; magnetic disks such as internal hard disks and removable disks; magneto-optical disks; and CD-ROM (compact disc read-only memory) and DVD-ROM (digital versatile disc read-only memory) disks. The processor and the memory can be supplemented by, or incorporated in, ASICs (application-specific integrated circuits).

To provide for interaction with a user, some features can be implemented on a computer having a display device such as a CRT (cathode ray tube) or LCD (liquid crystal display) monitor for displaying information to the user and a keyboard and a pointing device such as a mouse or a trackball by which the user can provide input to the computer.

In some embodiments, features described herein can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include, e.g., a LAN (local area network), a WAN (wide area network), and the computers and networks forming the Internet.

In some embodiments, the computer system can include clients and servers. A client and server are generally remote from each other and typically interact through a network, such as the described one. The relationship of client and server arises by virtue of computer programs running on the respective computers and having a client-server relationship to each other.

### Other Embodiments

It is to be understood that while the invention has been described in conjunction with the detailed description thereof, the forgoing description is intended to illustrate and not limit the scope of the invention, which is defined by the scope of the appended claims. Other aspects, advantages, and modifications are within the scope of the following claims.

What is claimed is:

1. A method for measuring a component of a non-homogenized solution, the method comprising:
receiving, at a computer system, a datastream of sensed infrared energy generated by irradiating a sample of the non-homogenized solution with infrared energy, and sensing the infrared energy emitted from the irradiated sample;
determining, by the computer system, at least one of an infrared absorption spectrum and an infrared emission spectrum based on the datastream;
determining, by the system, a measured value of the component in the sample based on the one or more determined spectrum;
generating, by the computer system, an adjustment factor based on the one or more determined spectrum;
adjusting the measured value based on the adjustment factor to generate an adjusted value;
determining, by the system, a correction factor based on a selected particle size or particle scatter associated with the component in the sample;
modifying, by the computer system, the adjusted value using the correction factor, to generate a corrected value of the component; and,
outputting, by the computer system, information identifying the corrected value of the component, wherein the corrected value indicates a measurement of the component in the non-homogenized solution.

2. The method of claim 1, wherein the selected particle size is based on a mean particle size of a predetermined proportion of particles of the component in at least a portion of the sample.

3. The method of claim 2, wherein the mean particle size or particle scatter is determined based on at least one of the infrared absorption spectrum and the infrared emission spectrum.

4. The method of claim 1, wherein the selected particle size is determined based on a predetermined proportion of particles of the component in at least a portion of the sample having a mean diameter less than the selected particle size.

5. The method of claim 4, wherein the predetermined proportion is between twenty percent and one hundred percent.

6. The method of claim 5, wherein the predetermined proportion is ninety percent.

7. The method of claim 1, wherein the correction factor is based on a relationship between an error measurement model and the selected particle size or the particle scatter, where such relationship corresponds to a reference model associated with the component.

8. The method of claim 7, wherein the relationship is a linear relationship.

9. The method of claim 1, wherein generating an adjustment factor based on the one or more determined spectrum further comprises obtaining a reference spectrum associated with the component and comparing the one or more determined spectrum to the reference spectrum to generate the adjustment factor.

10. The method of claim 9, wherein comparing the one or more determined spectrum to the reference spectrum comprises performing a linear least squares regression.

11. The method of claim 9, wherein the reference spectrum is associated with measurements of the component in a homogenized solution.

12. The method of claim 1, wherein the non-homogenized solution is non-homogenized milk and the component is fat.

13. The method of claim 12, wherein generating an adjustment factor based on the one or more determined spectrum further comprises obtaining a reference spectrum associated with the component and comparing the determined spectrum to the reference spectrum to generate the adjustment factor wherein the reference spectrum is based on one or more mathematical models comprising Fat A model, Fat B model, Fat C model, Fat D model, or Fat PLS model.

14. The method of claim 13, wherein the correction factor is based on a relationship between error measurement and particle size, wherein the relationship is associated with a fat model spectrum comprising at least one of a Fat A model, a Fat B model, a Fat C model, a Fat D model, or a Fat PLS model.

15. The method of claim 14, wherein particle size is based on a mean diameter of at least some of the particles in the solution.

16. The method of claim 15, wherein the particle size is D₉₀.

17. The method of claim 1, wherein the sample is a solution of non-homogenized milk, and the component includes one or more of fat, protein, total protein, true protein, lactose, non-protein nitrogen, solids, or non-fat solids, or any combinations thereof.

18. A method for determining fat content of a non-homogenized solution, the method implemented by a system comprising at least one computer, the method comprising:
receiving a datastream of sensed infrared energy generated by irradiating a sample of the non-homogenized solution and sensing the infrared energy from the irradiated sample;
determining, at least one of an infrared absorption spectrum and an infrared emission spectrum based on the received datastream of sensed infrared energy;
selecting a reference spectrum for fat content based on the non-homogenized solution;
comparing at least one of the infrared absorption spectrum and the infrared emission spectrum to the reference spectrum to determine an adjusted value of an amount of fat in the non-homogenized solution;
determining a particle size of the fat content based on at least one of the infrared absorption spectrum and the infrared emission spectrum;
based on the determined particle size, computing a correction factor; and
determining the fat content in the sample by applying the correction factor to the adjusted value.

19. The method of claim 18, wherein determining a particle size comprises determining a fat particle size within a predetermined proportion of fat particles in at least a portion of the sample.

20. The method of claim 19, wherein the predetermined proportion is ninety percent.

21. The method of claim 18, wherein selecting a reference spectrum includes selecting at least one fat model spectrum associated with a Fat A model, a Fat B model, a Fat C model, a Fat D model, and a Fat PLS model.

22. The method of claim 21, wherein computing a correction factor includes selecting an error measurement model associated with the at least one selected fat model spectrum.

23. The method of claim 18, wherein the non-homogenized solution is one of an animal dairy product or a non-animal milk product.

24. The method of claim 23, wherein the animal dairy product comprises at least one of raw milk, milk, cream, ice cream, yogurt, cheese, or any combinations thereof.

25. The method of claim 23, wherein the animal dairy product comprises milk from at least one of a cow, a sheep, a camel, a buffalo, a goat, and a human.

26. A system for sensing a property of a non-homogenized solution containing one or more components, the system comprising:
a sample chamber configured to receive a sample of the non-homogenized solution;
an infrared energy source configured to, when energized, irradiate the sample with infrared energy;
an infrared sensor comprising:
a sensing element positioned to receive infrared energy emitted from the irradiated sample and configured to generate a datastream based on the received infrared energy; and
a controller comprising a processor and a memory, the controller being in data communication with the infrared sensor, the controller configured to:
determine at least one of an infrared absorption spectrum and an infrared emission spectrum from the datastream;
process the one or more measured spectrum to compute a measured value of an amount of a component of the sample;
process the one or more determined spectrum to generate an adjustment factor;
adjust the measured value based on the adjustment factor to generate an adjusted value;
determine a correction factor based on a selected particle size associated with the component in the sample; and,
modify the adjusted value using the correction factor, to generate a corrected value of the component.

27. The system of claim 26, wherein the controller is further configured to:
compare the corrected value of the component to a ruleset to identify an operation defined by the ruleset; and,
responsive to the identification of an operation, issue a command to cause the operation to occur.

28. The system of claim 27, wherein the operation comprises at least one of: initiating operation of a device that manufactures a product using the non-homogenized solution; actuating a transfer device that transfers the non-homogenized solution from an initial location to a destination location; transmitting a first data record over a data network, the data record created based on the corrected value; and causing storing of a second data record to a computer-readable destination.
